(19) United States
(12) Patent Application Publication
(10) Pub. No.: US 2025/0169828 A1
(43) Pub. Date: May 29, 2025

(54) CUT GUIDE WITH INTEGRATED JOINT REALIGNMENT FEATURES

(71) Applicant: Treace Medical Concepts, Inc., Ponte Vedra, FL (US)

(72) Inventors: Adriaan Kuyler, Ponte Vedra, FL (US); Jody McAleer, Jefferson City, MO (US); William DeCarbo, Pittsburgh, PA (US); Robert Santrock, Morgantown, WV (US); Sean Scanlan, Jacksonville, FL (US)

(21) Appl. No.: 19/019,429

(22) Filed: Jan. 13, 2025

Related U.S. Application Data
(63) Continuation of application No. 17/750,275, filed on May 20, 2022, now Pat. No. 12,193,683.
(60) Provisional application No. 63/246,792, filed on Sep. 21, 2021.
(60) Provisional application No. 63/190,787, filed on May 20, 2021.

(51) Int. Cl. A61B 17/15; A61B 17/16; A61B 17/17; A61B 17/56; A61B 17/66

(52) U.S. Cl. CPC A61B 17/151; A61B 17/1682; A61B 17/1775; A61B 17/66; A61B 2017/565

(57) ABSTRACT

A bone cutting and joint realignment instrument may include an integrated spacer body, bone preparation guide, and fulcrum body. The spacer body is configured to be inserted into a joint space between a metatarsal and an opposed cuneiform of a foot. The bone preparation guide body is affixed to the spacer body with the spacer body extending downwardly from the bone preparation guide body. The bone preparation guide body can define at least one guide surface configured to be positioned over at least one of the metatarsal and the opposed cuneiform. The fulcrum body may be rotatably coupled to the spacer body within a bounded range of rotation, such as a bounded range of less than 45 degree. The fulcrum body can be configured to be inserted in an intermetatarsal space between the metatarsal and an adjacent metatarsal.

### CROSS-REFERENCE

This application is a continuation of U.S. patent application Ser. No. 17/750,275, filed May 20, 2022, and issued as U.S. Pat. No. 12,193,683 on Jan. 14, 2025, which claims the benefit of U.S. Provisional Patent Application No. 63/246,792, filed Sep. 21, 2021, and U.S. Provisional Patent Application No. 63/190,787, filed May 20, 2021. The entire contents of each of these applications are incorporated herein by reference.

### TECHNICAL FIELD

This disclosure relates to surgical devices and, more particularly, to surgical devices for assisting in bone cutting and/or realignment techniques.

### BACKGROUND

Bones within the human body, such as bones in the foot, may be anatomically misaligned. For example, one common type of bone deformity is hallux valgus, which is a progressive foot deformity in which the first metatarsophalangeal joint is affected and is often accompanied by significant functional disability and foot pain. The metatarsophalangeal joint is medially deviated, resulting in an abduction of the first metatarsal while the phalanges adduct. This often leads to development of soft tissue and a bony prominence on the medial side of the foot, which is called a bunion.

Surgical intervention may be used to correct a bunion deformity. A variety of different surgical procedures exist to correct bunion deformities and may involve removing the abnormal bony enlargement on the first metatarsal and/or attempting to realign the first metatarsal relative to the adjacent metatarsal. Surgical instruments that can facilitate efficient, accurate, and reproducible clinical results are useful for practitioners performing bone realignment techniques.

### SUMMARY

In general, this disclosure is directed to a combined bone cutting and joint realignment instrument that includes an integrated spacer body, bone preparation guide, and fulcrum body. The spacer body is configured to be inserted into a joint space between a metatarsal and an opposed cuneiform of a foot. The bone preparation guide body can be affixed to the spacer body with the spacer body extending downwardly from the bone preparation guide body. The bone preparation guide body can define at least one guide surface configured to be positioned over at least one of the metatarsal and the opposed cuneiform. The fulcrum body may be rotatably coupled to the spacer body within a bounded range of rotation, such as a bounded range of less than 45 degree. The fulcrum body can be configured to be inserted in an intermetatarsal space between the metatarsal and an adjacent metatarsal.

In practice, the combined bone cutting and joint realignment instrument can be used in a bone preparation and/or realignment procedure, such as a hallux valgus or bunion correction procedure performed on the first metatarsal of the foot. The spacer of the combination instrument may be inserted into the joint space between the first metatarsal and medial cuneiform. With the integrated bone preparation guide body attached thereto, the step of positioning the spacer body can simultaneously position one or more guide surfaces defined by the bone preparation guide over one or more bones (e.g., the metatarsal and/or cuneiform) to be prepared using the guide. In addition, the fulcrum body connected to the spacer body can be simultaneously positioned in the intermetatarsal space between the lateral side of the first metatarsal and medial side of an adjacent second metatarsal.

By configuring the combined instrument with a rotatable connection between the spacer body and fulcrum body, the clinician may have flexibility to manipulate the relative positions of the spacer body and fulcrum body, and bone preparation guide body associated therewith, relative to each other. For example, the spacer body may be freely rotatable relative to the fulcrum body within a bounded range of travel. This can establish a floppy connection between the two components, allowing the clinician to easily manipulate the position of the bone preparation guide body relative to one or more bones to be cut, e.g., causing the spacer body and fulcrum body to move relative to each other about the rotatable connection as the bone preparation guide body is moved. Further, by limiting the range of rotation between the spacer body and fulcrum body, the relative movement between the two components may be limited so the components do not get so far out of alignment with each other as to be challenging for the surgeon to work with the instrument.

To provide a combined instrument, the bone preparation guide body can be affixed to the spacer body and/or fulcrum body. For example, the bone preparation guide body can be permanently fabricated with the spacer body and/or fulcrum body (e.g., through casting, milling, machining, molding, 3D printing) to form a monolithic structure or fabricated separately and subsequently attached thereto to provide an integrated, unitary instrument comprised of inseparable features under normal use. In either case, the bone preparation guide body is affixed to the spacer body and/or fulcrum body so as to define a unitary instrument for use during a surgical procedure.

Providing a combined instrument having both spacer, fulcrum, and bone preparation guide functionalities can be exceptionally useful during a surgical procedure. Access to a patient's subcutaneous bone structure is typically limited by a comparatively small incision. Combining different functionalities into a unitary instrument can allow the instrument to be effectively and rapidly positioned through the small incision. Moreover, the surgeon and support staff have a limited number of hands with which to manipulate different tools and instruments used during the surgical procedure. Providing a combined instrument can allow a clinician to rapidly deploy the instrument during a surgical procedure, reducing the duration of the procedure and allowing more effective utilization of surgical resources.

The bone preparation guide body associated with the combined instrument can have a number of different forms. In general, the bone preparation guide body defines at least one guide surface positionable over a metatarsal and/or opposed cuneiform. A clinician can guide a tissue removal instrument along the guide surface (e.g., abutting the guide surface) to guide removal of an end portion of the metatarsal and/or opposed cuneiform. For example, the bone preparation guide body may define a first guide surface positionable over a metatarsal and a second guide surface positionable over the opposed cuneiform, with the two guide surfaces being separated from each other by a distance sufficient to span the tarsometatarsal joint. In various examples, the one or more guide surfaces include a facing guide surface to define a cutting slot there between.

In use, the clinician may position the bone preparation guide body over a dorsal surface of one or more bones to be prepared (e.g., a metatarsal and/or cuneiform). The clinician can then check the alignment of one or more guide surfaces defined by the bone preparation guide relative to the one or more bones to confirm the amount and/or orientation of bone to be removed (e.g., cut to be made) before making a cut. The clinician may perform this check visually with the unaided eye and/or through X-Ray imaging (fluoroscopy). The clinician can adjust the position of the combined instrument, particularly the bone preparation guide body, until the one or more guide surfaces are in a desired position and/or alignment. The clinician may insert one or more fixation pins through the bone preparation guide body to temporarily fixate the desired position before making one or more cuts using the guide body.

The bulk of the combination instrument, including spacer body, fulcrum body, and bone preparation guide body may have a tendency to obscure visualization (e.g., fluoroscopy) of the one or more guide surfaces relative to the underlying bones to be prepared using the bone preparation guide body. To reduce or eliminate this obscuring, the bone preparation guide body may be configured with a comparatively open framework. For example, the bone preparation guide body and/or guide surfaces defined by the body may be formed of comparatively thin-walled materials. This can allow the clinician to see around and/or through the walls of the bone preparation guide body (e.g., during imaging) to facilitate accurate aligning of the one or more guide surfaces relative to one or more bones to be cut. For example, the walls of the bone preparation guide body may have a thickness (e.g., in a proximal to distal direction along the length of the bone being cut when positioned over the dorsal surface of the bone) less than 2 mm, such as less than 1.5 mm, or less than approximately 1.0 mm (for example, ±10 percent).

In one example, a bone cutting and joint realignment instrument is described that includes a spacer body, bone preparation guide body, and fulcrum body. The spacer body is configured to be inserted into a joint space between a metatarsal and an opposed cuneiform of a foot. The bone preparation guide body is affixed to the spacer body with the spacer body extending downwardly from the bone preparation guide body. The bone preparation guide body defines at least one guide surface configured to be positioned over at least one of the metatarsal and the opposed cuneiform. The fulcrum body is rotatably coupled to the spacer body within a bounded range of rotation (e.g., less than 45 degrees). The fulcrum body is configured to be inserted in an intermetatarsal space between the metatarsal and an adjacent metatarsal.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIGS. 4A and 4B are perspective and top views, respectively, of an example bone positioning operation in which a combined instrument is positioned in a first joint space and an intersecting second joint space.

FIGS. 5A and 5B are perspective and top views, respectively, of an example configuration of a combined instrument.

FIGS. 6A and 6B illustrate one example coupling arrangement that can be used to connect a spacer body to a fulcrum body on a combined instrument according disclosure.

FIGS. 7A and 7B are top sectional views of a combined instrument showing example bounded rotational positions to which a fulcrum body and a spacer body can rotate.

FIGS. 8A and 8B illustrate two example configurations of a combined instrument including an actuator/locking feature.

### DETAILED DESCRIPTION

This disclosure is generally directed to a combined bone cutting and joint realignment instrument that includes an integrated spacer body, bone preparation guide, and fulcrum body. The spacer body is configured to be inserted into a joint space between a metatarsal and an opposed bone (e.g., cuneiform, cuboid) of a foot. The bone preparation guide body can be affixed to the spacer body with the spacer body extending downwardly from the bone preparation guide body. The bone preparation guide body can define at least one guide surface configured to be positioned over at least one of the metatarsal and the opposed cuneiform. The fulcrum body may be rotatably coupled to the spacer body, e.g., within a bounded range of rotation. The fulcrum body can be configured to be inserted in an intermetatarsal space between the metatarsal and an adjacent metatarsal.

The combined instrument can be used in a surgical procedure where one or more bone portions are to be prepared, such as a bone realignment procedure. Example procedures in which the combined instrument can be used include a bone alignment, osteotomy, fusion procedure, and/or other procedures where one or more bones are operated upon and/or realigned relative to one or more other bones. Such a procedure can be performed, for example, on bones (e.g., adjacent bones separated by a joint or different portions of a single bone) in the foot or hand, where bones are relatively smaller compared to bones in other parts of the human anatomy.

In one example, a procedure utilizing a combined instrument can be performed to correct an alignment between a metatarsal (e.g., a first metatarsal) and a second metatarsal and/or a cuneiform (e.g., a medial, or first, cuneiform), such as in a bunion correction surgery. An example of such a procedure is a Lapidus procedure (also known as a first tarsal-metatarsal fusion). In other examples, a procedure utilizing a combined instrument can be performed to correct an alignment of a lesser metatarsal (e.g., second, third, four, or fifth metatarsal). As still a further example, a procedure utilizing a combined instrument can be performed as part of a metatarsal-phalange arthrodesis procedure to correct a relative position of a metatarsal and proximal phalanx across a metatarsophalangeal joint. While the example instruments of the disclosure are generally described as being useful for insertion into a space between opposed bone ends transitioning into an intermetatarsal space, the instruments may be used in any desired application and the disclosure is not limited in this respect.

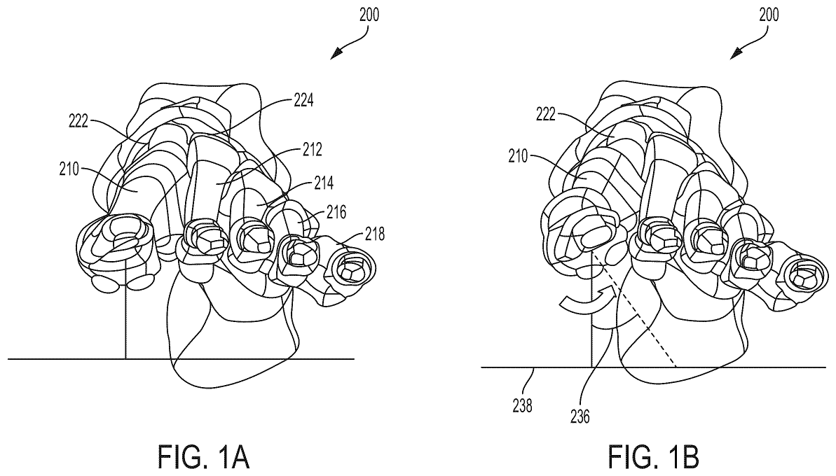

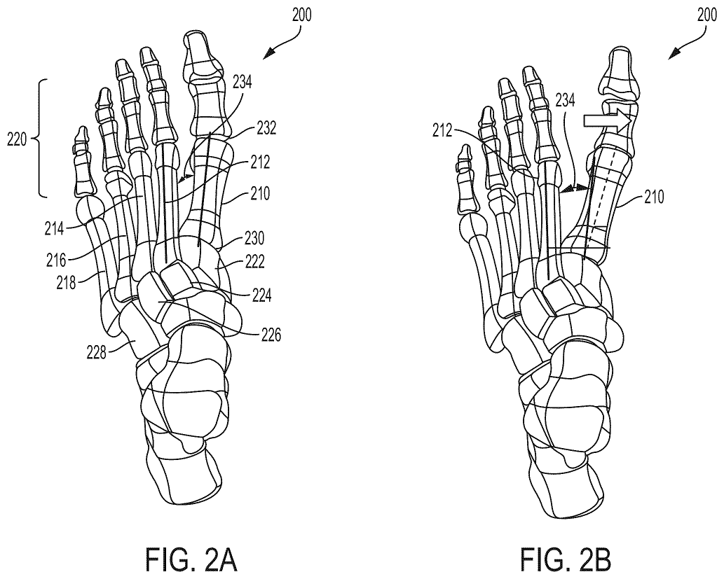

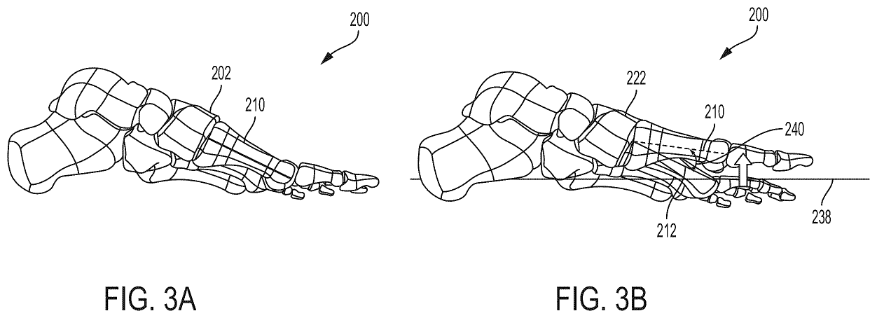

FIGS. 1-3 are different views of a foot 200 showing example anatomical misalignments that may occur and be corrected using a combined bone cutting and joint realignment instrument according to the present disclosure. Such misalignment may be caused by a hallux valgus (bunion), natural growth deformity, or other condition causing anatomical misalignment. FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane misalignment in each of FIGS. 1B, 2B, and 3B is for purposes of illustration and a metatarsal may be misaligned in multiple planes that is desirably corrected.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. The first metatarsal 210 is connected proximally to a medial cuneiform 222, while the second metatarsal 212 is connected proximally to an intermediate cuneiform 224, and the third metatarsal is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The joint 232 between a metatarsal and respective proximal phalanx is referred to as a metatarsophalangeal (“MTP”) joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and is perpendicular to an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

A combined instrument according to the disclosure can define a spacer body extending a medial-to-lateral direction (e.g., parallel to the frontal plane) of the foot that is coupled to a fulcrum body extending in a proximal to distal direction (e.g., parallel to sagittal plane) of the foot. An integrally affixed bone preparation guide body may be carried by the spacer body such that the bone preparation guide body is positioned over one or more bones to be cut by positioning the spacer body in a joint space (e.g., TMT joint space) between a metatarsal and opposed cuneiform while the fulcrum body is simultaneously positioned in an intermetatarsal space between the metatarsal facing the spacer body and an adjacent metatarsal. The combined instrument can be used as part of a bone positioning technique to correct an anatomical misalignment of a bone or bones.

In some applications, the technique involves realigning a metatarsal relative to an adjacent cuneiform and/or adjacent metatarsal. The metatarsal undergoing realignment may be anatomically misaligned in the frontal plane, transverse plane, and/or sagittal plane, as illustrated and discussed with respect to FIGS. 1-3 above. Accordingly, realignment may involve releasing the misaligned metatarsal or portion thereof for realignment and thereafter realigning the metatarsal in one or more planes, two or more planes, or all three planes. After suitably realigning the metatarsal, the metatarsal can be fixated to hold and maintain the realigned positioned.

While a metatarsal can have a variety of anatomically aligned and misaligned positions, in some examples, the term “anatomically aligned position” means that an angle of a long axis of first metatarsal 210 relative to the long axis of second metatarsal 212 is about 10 degrees or less in the transverse plane and/or sagittal plane. In certain embodiments, anatomical misalignment can be corrected in both the transverse plane and the frontal plane. In the transverse plane, a normal IMA 234 between first metatarsal 210 and second metatarsal 212 is less than about 9 degrees. An IMA 234 of between about 9 degrees and about 13 degrees is considered a mild misalignment of the first metatarsal and the second metatarsal. An IMA 234 of greater than about 16 degrees is considered a severe misalignment of the first metatarsal and the second metatarsal.

In some applications, a combined instrument is used as part of a realignment technique to anatomically align first metatarsal 210 or a portion thereof by reducing the IMA from over 10 degrees to about 10 degrees or less (e.g., to an IMA of about 1-5 degrees), including to negative angles of about −5 degrees or until interference with the second metatarsal, by positioning the first metatarsal at a different angle with respect to the second metatarsal.

With respect to the frontal plane, a normal first metatarsal will be positioned such that its crista prominence is generally perpendicular to the ground and/or its sesamoid bones are generally parallel to the ground and positioned under the metatarsal. This position can be defined as a metatarsal rotation of 0 degrees. In a misaligned first metatarsal, the metatarsal is axially rotated between about 4 degrees to about 30 degrees or more. In some embodiments, a combined instrument is used as part of a realignment technique to anatomically align the metatarsal by reducing the metatarsal rotation from about 4 degrees or more to less than 4 degrees (e.g., to about 0 to 2 degrees) by rotating the metatarsal with respect to the medial cuneiform.

A combined instrument that defines a spacer body rotatably coupled to a fulcrum body and a bone preparation guide body integrally affixed to the instrument (e.g., to the spacer body and/or fulcrum body) according to the disclosure may be useful to provide a unitary structure (e.g., prior to or after being assembled) that can be positioned between two adjacent, intersecting joint spaces—a first joint space between opposed ends of a metatarsal and cuneiform and an intermetatarsal space between adjacent metatarsals- and then used to cut one or both ends of the metatarsal and/or cuneiform defining the first joint space. The spacer body can include a portion insertable into the joint space to locate the bone preparation guide affixed thereto relative to the joint space and, correspondingly, the bones defining the joint space. The fulcrum body can establish and/or maintain space between adjacent bones being moved, e.g., helping to prevent lateral translation or base shift of the bones during rotation and/or pivoting.

For example, the combine instrument can include a spacer body positionable in the joint space between first metatarsal 210 and medial cuneiform 222 with a bone preparation guide body affixed thereto. The bone preparation guide body may include at least one guide surface, such as a cutting slot, positioned over an end of first metatarsal 210 and/or an end of medial cuneiform 222 to be cut, such as at least one metatarsal side guide surface (e.g., cutting slot) positionable over an end of first metatarsal 210 to be cut and at least one cuneiform side guide surface (e.g., cutting slot) positionable over an end of medial cuneiform 222 to be cut.

The combined instrument can also include a fulcrum body positionable in a joint space between first metatarsal 210 and second metatarsal 212. The fulcrum body can be inserted in the notch between first metatarsal 210 and second metatarsal 212 at the base of the metatarsals (e.g., adjacent respective cuneiforms) before moving the first metatarsal, e.g., to help avoid the proximal-most base of the first metatarsal 210 from shifting toward the proximal-most base of the second metatarsal 212. The fulcrum body can provide a point about which first metatarsal 210 can rotate and/or pivot while helping minimize or avoid base compression between the first metatarsal and the second metatarsal. In addition, use of the fulcrum body may cause first metatarsal 210 and medial cuneiform 222 to be better angled relative to one or more guide surfaces positioned over the end faces of the bones (one or more guide surfaces of the bone preparation guide body engaged with the spacer body), providing a better cut angle guided by the one or more guide surfaces than without use of the fulcrum body. This can help reduce or eliminate unwanted spring-back, or return positioning, of first metatarsal 210 after initial realignment of the metatarsal.

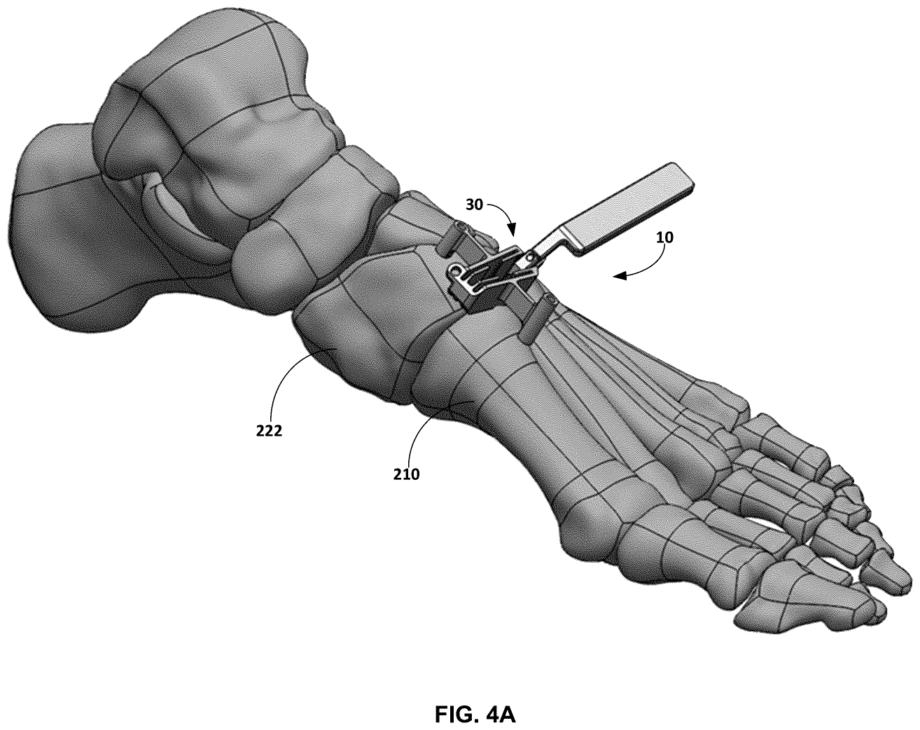

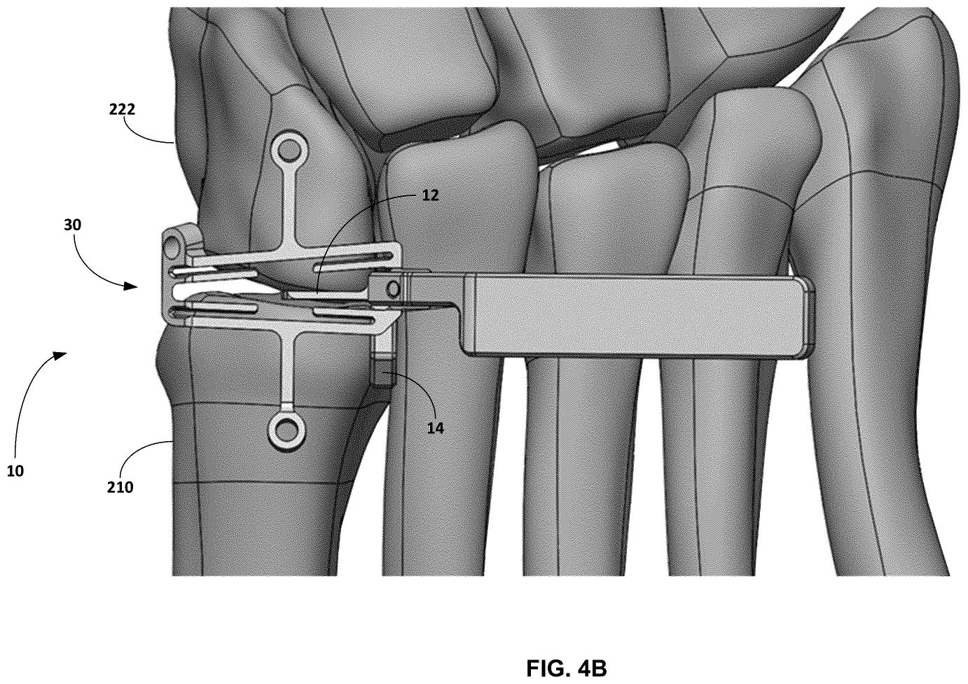

FIGS. 4A and 4B (referred to collectively as FIG. 4) are perspective and top views, respectively, of an example bone positioning operation in which a combined instrument 10 is positioned in a first joint space and an intersecting second joint space, where a bone forming the first and second joint spaces is being realigned relative to one or more adjacent bones. In particular, FIG. 4 illustrates a combined bone cutting and joint realignment instrument 10 having a spacer body 12 rotatably coupled to a fulcrum body 14 and a bone preparation guide body 30 affixed to the spacer body. Spacer body 12 is positioned at an intersection between an end of first metatarsal 210 and opposed medial cuneiform 222. Fulcrum body 14 is positioned between first metatarsal 210 and second metatarsal 212. Instrument 10 may optionally be used in conjunction with other surgical devices, such as a bone positioning guide that is operable to move first metatarsal 210 in one or more planes. Additional details on example bone positioning guides and related techniques are described in U.S. patent application Ser. No. 14/981,335, filed Dec. 28, 2015, and U.S. patent application Ser. No. 15/236,464, filed Aug. 14, 2016, the entire contents of which are incorporated herein by reference.

As shown in the example of FIG. 4, spacer body 12 can be positioned between opposed end of adjacent bones, such as opposed ends of a metatarsal (e.g., first metatarsal 210) and cuneiform (e.g., medial cuneiform 222) separated by a joint space. Spacer body 12 can define a length configured to be inserted into the joint space between the two bones, a thickness configured to extend between the metatarsal and the opposed cuneiform (e.g., with first metatarsal 210 and medial cuneiform 222 contacting opposed sides of the spacer body), and a width configured to extend in a medial to lateral direction across at least a portion of the joint space.

Spacer body 12 can be positioned at any suitable location across the joint space (e.g., in the front plane). In some examples, spacer body 12 extends across the entire width of the joint space between first metatarsal 210 and medial cuneiform 222, e.g., from a medial-most end of the joint space to a lateral-most end of the joint space. In other configurations, spacer body 12 extends across less than the entire width of the joint space, such as a lateral-most half or less of the joint space, or a lateral-most quarter or less of the joint space.

In some configurations, spacer body 12 has a length (in the dorsal to plantar direction) sufficient such that, when the spacer body is inserted into the joint space, the spacer body projects dorsally above the joint space. In other configurations, spacer body 12 may be comparatively smaller such that, when the spacer body is inserted into the joint space, the top edge of the spacer body is flush with or recessed relative to the dorsal-most surface of first metatarsal 210 and/or medial cuneiform 222 at the joint. This latter configuration can be useful to help prevent spacer body 12 from visually obstructing the joint space.

Bone preparation guide body 30 may affixed to spacer body 12 to define a unitary/integral instrument. The positioning of spacer body 12 in the joint space can dictate the positioning of bone preparation guide body 30 coupled thereto and, correspondingly, the guiding of a bone preparation instrument facilitated by the bone preparation guide.

Combined instrument 10 also includes fulcrum body 14. In use, the clinician can insert fulcrum body 14 between first metatarsal 210 and second metatarsal 212 (or other adjacent bones, when not performing a metatarsal realignment) concurrent with inserting spacer body 12 into the TMT joint space between first metatarsal 210 and medial cuneiform 222. For example, the clinician can insert spacer body 12 in the joint space between first metatarsal 210 and medial cuneiform 222 and also insert fulcrum body 14 in the joint space between first metatarsal 210 and second metatarsal 212 at the same time. Bone preparation guide body 30 affixed to instrument 10 is positioned over a dorsal side of first metatarsal 210 and/or medial cuneiform 222 concurrently upon insertion of the spacer body and fulcrum body into respective joint spaces.

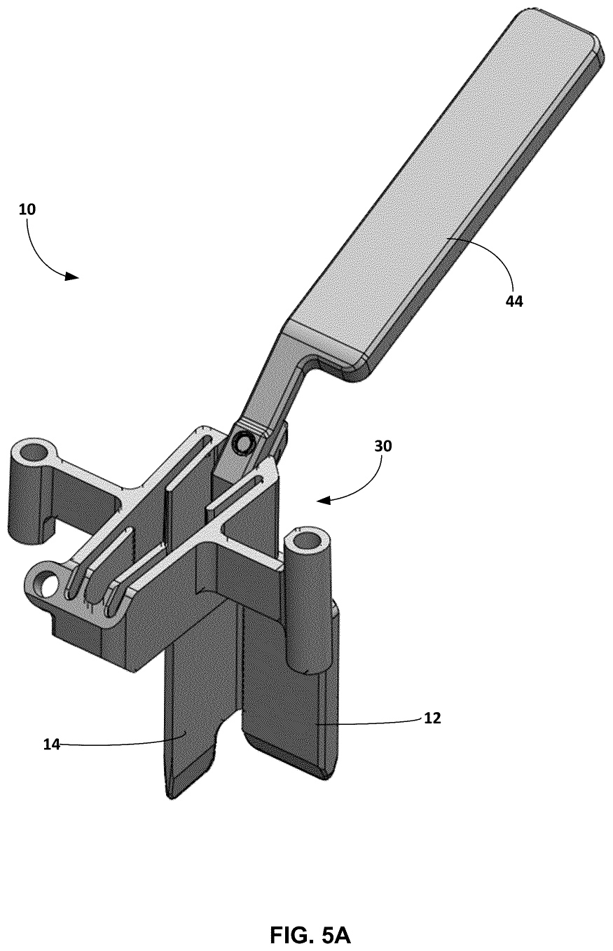

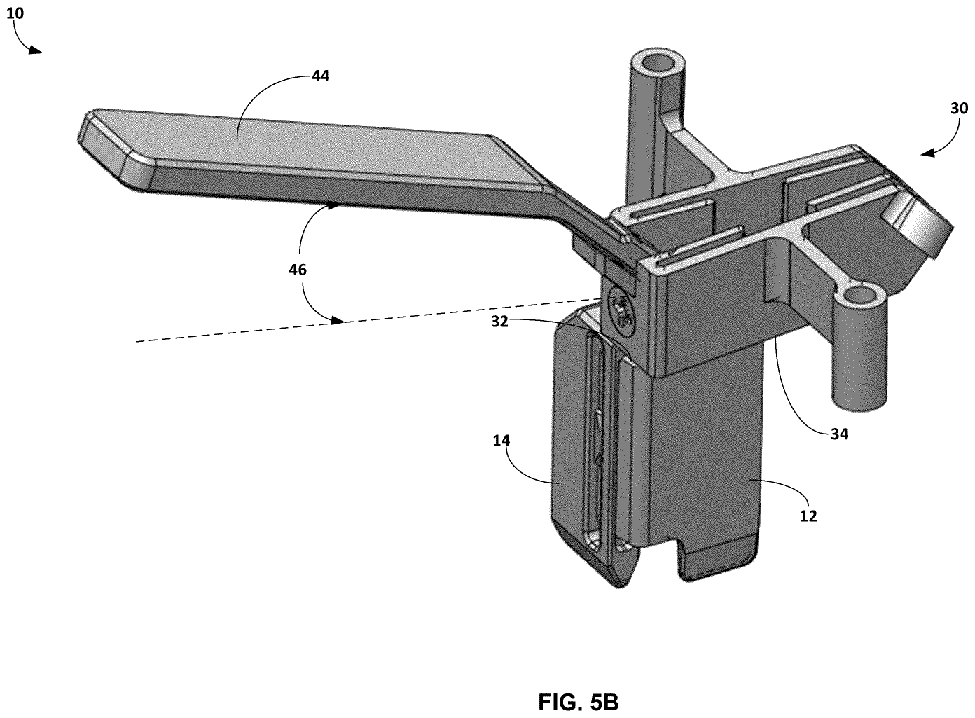

FIGS. 5A and 5B (collectively referred to as FIG. 5) are perspective and top views, respectively, of an example configuration of combined instrument 10. As shown in this example, instrument 10 includes bone preparation guide body 30 affixed to spacer body 12 and spacer body 12 rotatably coupled to fulcrum body 14.

In general, spacer body 12 may define a length configured to be inserted into the joint space, a thickness configured to extend between the bones defining the joint space (e.g., metatarsal 210 and opposed cuneiform 222), and a width configured to extend in a medial to lateral direction partially or fully across the joint space. Spacer body 12 can have a length extending from a top end to a bottom end. In some examples, bone preparation guide body 30 is affixed to spacer body 12 such that the top end of the spacer body is approximately coplanar with or located vertically below the bottom end of the bone preparation guide body (e.g., the bottom end of at least one guide surface of the bone preparation guide body). For example, as shown in FIG. 5, a top surface 32 of spacer body 12 may be coplanar with or recessed relative to a bottom edge 34 of bone preparation guide body 30.

Fulcrum body 14 can define a length configured to be inserted into the intermetatarsal space, a thickness configured to extend between first metatarsal 210 and second metatarsal 212, and a width configured to extend in the proximal to distal direction across the foot. The thickness of fulcrum body 14 may be substantially constant across the length of the fulcrum body or may be tapered toward the leading end to facilitate insertion of fulcrum body 14 into a space between adjacent metatarsals. In general, fulcrum body 14 may have a width that extends partially within the intermetatarsal space between first metatarsal 210 and second metatarsal 212. When inserted into the intermetatarsal space, fulcrum body 14 may extend from the base (e.g., proximal-most end) of first metatarsal 210 toward the distal-most end of the first metatarsal a distance less than half the length of the metatarsal, such as a distance less than a quarter of the length of the metatarsal, a distance less than 10% of the length of the metatarsal, or a distance less than 5% of the length of the metatarsal.

In some examples, instrument 10 includes a handle 44. Handle 44 can be operatively connected to and extend from bone preparation guide body 30. By connecting handle directly to bone preparation guide body 30, the clinician may more easily manipulate the location of one or more guide surfaces defined by bone preparation guide body 30 than if the handle is attached to another portion of instrument 10. Handle 44 may be any structure projecting from bone preparation guide body 30 of instrument 10 that can provide a gripping location for the instrument during use.

In some examples, such as the example illustrated in FIG. 5, handle 44 can project angularly away from bone preparation guide body 30 to define a tissue retraction space 46. The tissue retraction space may be a region bounded on one side by bone preparation guide body 30 and one side of handle 44. In use, combined instrument 10 may be inserted into an incision space with handle 44 extending out of the surgical incision and over an epidermal layer with tissue captured in the tissue retraction space. For example, combined instrument 10 may be inserted into an intermetatarsal space with handle 44 projecting toward the lateral side of the foot being operated upon. The tissue retraction space may help retract tissue and push the tissue laterally away from a first metatarsal and/or medial cuneiform being operated upon. In other configurations, however, handle 44 may extend at a different direction (e.g., straight upwardly or dorsally from the bone preparation guide body) and/or be attached to a different portion of instrument 10 (e.g., fulcrum body 14) then bone preparation guide body 30. In still other configurations, instrument 10 may not include a handle 44.

Fulcrum body 14 is operatively coupled to spacer body 12. In some configurations, fulcrum body 14 is fixedly coupled to spacer body 12 to form a permanent, unmovable connection between the fulcrum body and spacer body. In other examples, however, fulcrum body 14 may be movably coupled to spacer body 12 such that the fulcrum body is rotatable relative to the spacer body. Configuring fulcrum body 14 to be relatively rotatable to spacer body 12 can be useful to allow the angle between the fulcrum body and spacer body to be changed or manipulated by the clinician to accommodate different patient anatomies and conditions that may be encountered during a particular surgical procedure.

In some implementations, fulcrum body 14 is rotatably coupled to spacer body 12 within a bounded range of rotation. That is, fulcrum body 14 may be mechanically coupled to spacer body 12 to provide a unitary instrument but may be rotatable relative to the spacer body within a constrained or limited range of rotation. Limiting the range of rotation between fulcrum body 14 and spacer body 12 can be beneficial to allow some relative movement between the spacer body and fulcrum body but not providing too much relative rotation such that the fulcrum body becomes overly floppy or difficult for the clinician to manipulate during a surgical procedure.

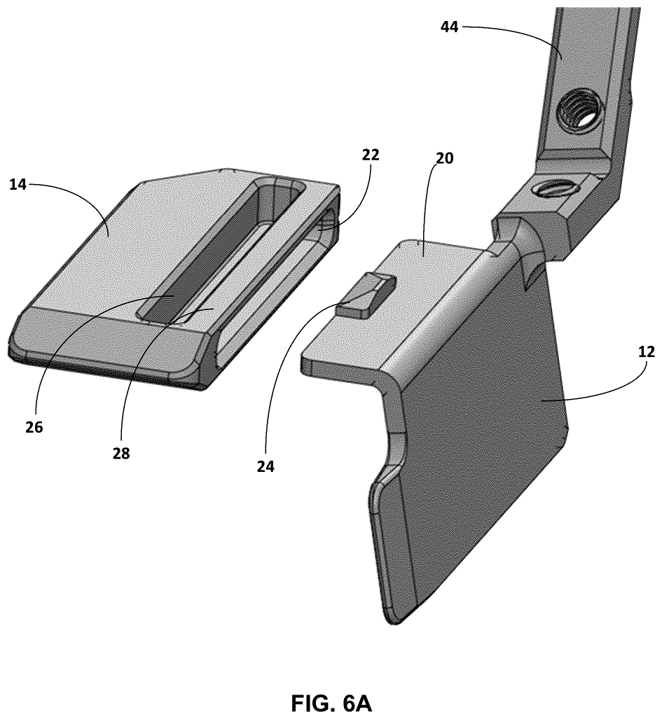

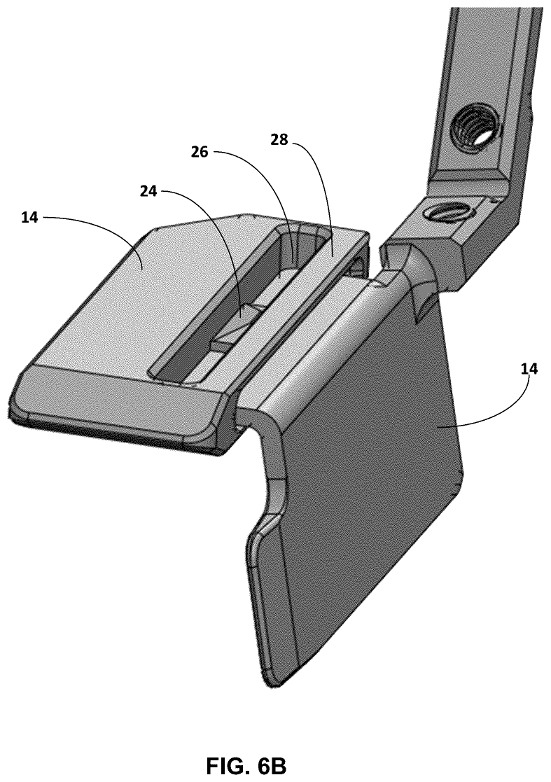

While various coupling arrangements may be used to operatively connect fulcrum body 14 to spacer body 12, FIGS. 6A and 6B (collectively referred to as FIG. 6) illustrate one example coupling arrangement that can be used to connect a spacer body to a fulcrum body on a combined instrument according disclosure. As shown in the illustrated example, a complementary connection between fulcrum body 14 and spacer body 12 can be provided in which one of the fulcrum body and spacer body has a projecting tongue 20 and the other of the fulcrum body and the spacer body has a corresponding receiving groove 22 into which the projecting tongue 20 can be inserted. Projecting tongue 20 can extend at an angle relative to the plane defined by the widthwise direction of fulcrum body 14 or spacer body 12, such as an angle approximately orthogonal (e.g., 80° to 100°, such as 85° to 95°, or from 88° to 92°, or 90°) to the plane defined by the widthwise direction of the fulcrum body or spacer body.

In the illustrated arrangement, projecting tongue 20 is illustrated as extending orthogonally from spacer body 12. When spacer body 12 is positioned in a tarsometatarsal joint space extending in a medial-to-lateral direction, projecting tongue 20 may extend in a proximal-to-distal direction in the intermetatarsal space between first metatarsal 210 and second metatarsal 212. Projecting tongue 20 may have a width (e.g., in the proximal-to-distal direction when inserted into the joint) less than the width of spacer body 12, such as a width less than or equal to three quarters the width of the spacer body, less than or equal to half the width of the spacer body, or less than or equal to one fifth of the width of the spacer body.

Receiving groove 22 can define an opening into which projecting tongue 20 can be inserted. Receiving groove 22 is illustrated as extending in the widthwise direction across a portion of the width of fulcrum body 14. When so configured, receiving groove 22 can extend in the proximal-to-distal direction in the intermetatarsal space between first metatarsal 210 and second metatarsal 212, when fulcrum body 14 is inserted into the metatarsal space.

Receiving groove 22 can define an opening cross-sectional size larger than a cross-sectional size of projecting tongue 20 to allow the tongue to rotate relative to the groove. To retain projecting tongue 20 in receiving groove 22, one of the tongue and the groove can include a locking projection 24 and the other of the tongue and the groove can include a complementary locking receiving cavity 26.

For example, the locking projection 24 may extend outwardly from a face of the projecting tongue, and the complementary locking receiving cavity 26 may extends through a wall surface of the other of the spacer body and the fulcrum body defining the groove. Locking receiving cavity 26 may be configured (e.g., sized and/or shaped) for locking projection 24 to extend partially or fully into and/or through the locking receiving cavity 26.

Locking projection 24 may be a region of thicker material extending perpendicularly outwardly relative to the face of projecting tongue 20 (e.g., in a direction perpendicular to the width and length of the projecting tongue). Locking projection 24 can have any polygonal or arcuate shape and, in some examples, may have a tapered profile from a comparatively narrower end advanced in a leading direction into receiving groove 22 to a comparatively wider end advanced in a trailing direction into the receiving groove.

When locking projection 24 extends generally perpendicularly relative to the face of projecting tongue 20 (e.g., defining a portion of increased thickness), locking receiving cavity 26 may define an opening extending through a wall surface that is perpendicular to the wall surface defining receiving groove 22. In some examples, a beam 28 extends between top and bottom wall surfaces of fulcrum body 14, bounding receiving groove 22 on one side and locking receiving cavity 26 on another side. Beam 28 may be sufficiently thin and/or flexible so as to elastically bend upon insertion of projecting tongue 20 into receiving groove 22 and then returned to an unbent shape to retain locking projection 24 in locking receiving cavity 26.

While the foregoing discussion of a rotatable connection between spacer body 12 and fulcrum body 14 has focused on a configuration in which projecting tongue 20 is formed on spacer body 12 and receiving groove 22 is defined on fulcrum body 14, projecting tongue 20 may alternatively extend from fulcrum body 14 and receiving groove 22 be defined by spacer body 12. Other examples of rotatable connections that may be used include a hinged connection between spacer body 12 and fulcrum body 14, e.g., with stops built in that limit the range of rotation between the spacer body and fulcrum body.

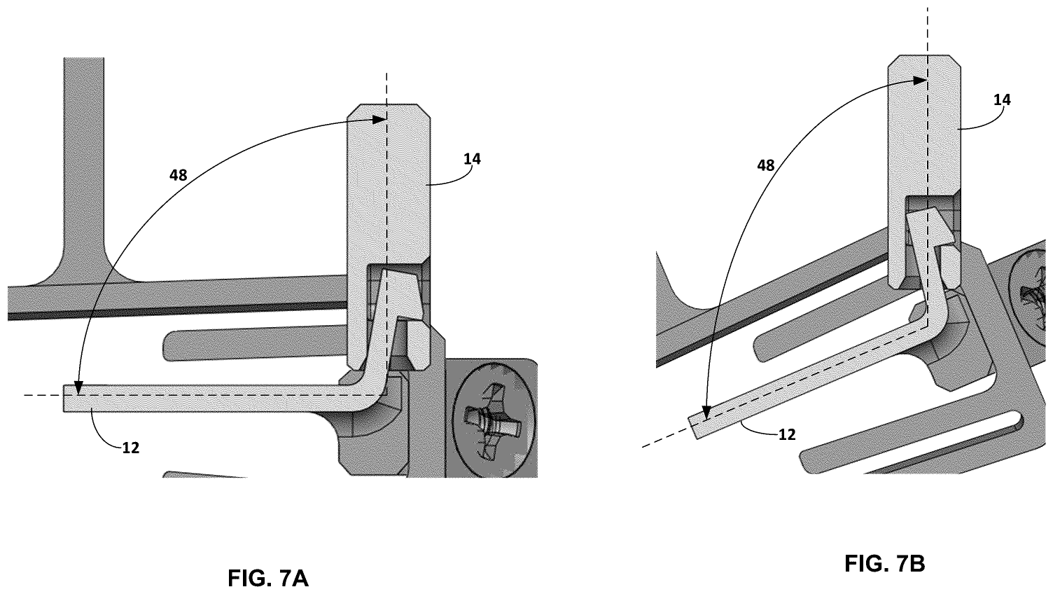

FIGS. 7A and 7B (collectively referred to as FIG. 7) are top sectional views of combined instrument 10 showing example bounded rotational positions to which fulcrum body 14 and spacer body 12 can rotate. FIG. 7 illustrates a combined instrument 10 utilizing the complementary connection features between spacer body 12 and fulcrum body 14 illustrated and described with respect to FIG. 6. However, the relative rotational positions and angles illustrated and described with respect to FIG. 7 may be achieved using other bounded rotational connection configurations between spacer body 12 and fulcrum body 14.

As shown in FIG. 7, spacer body 12 and fulcrum body 14 can define an angle of 48 between a plane defined by the widthwise extent of the spacer body and a plane defined by the widthwise extent of the fulcrum body. FIG. 7A illustrates a rotational position between spacer body 12 and fulcrum body 14 defining an example minimum intersection angle 48. FIG. 7B illustrates a rotational position between spacer body 12 and fulcrum body 14 defining an example maximum intersection angle 48. As shown in these examples, the amount of angular rotation between spacer body 12 and fulcrum body 14 may be controlled by the size of locking projection 24 and the corresponding size (e.g., depth) of locking receiving cavity 26.

In some examples, spacer body 12 and fulcrum body 14 are configured to rotate relative to each other to allow the angular intersection 48 between the two bodies to vary within a from 80 degrees to 125 degrees, optionally including all sub-ranges therein. In some examples, spacer body 12 and fulcrum body 14 are rotatably coupled together to allow angle 48 to adjust from a minimum of approximately 90 degrees (e.g., ±10%) to a greater angle. For example, spacer body 12 and fulcrum body 14 may be rotatably coupled together to allow angle 48 to adjust from a minimum of approximately 90 degrees to 120 degrees or less. For instance, in certain implementations, spacer body 12 and fulcrum body 14 may be rotatably coupled together to allow angle 48 to vary within a bounded range of rotation of approximately ±45° or less, such as ±35° or less, ±30° or less, ±25° or less, ±15° or less, or ±10° or less. The absolute value of angle 48 may be within a range from 90° to 115°, optionally including all sub-ranges therein.

In some examples, instrument 10 (e.g., spacer body 12, fulcrum body 14, bone preparation guide body 30) will be formed as a unitary structure, e.g., by milling, casting, or molding the components to be permanently and structurally integrated together. In other examples, one or more the features may be fabricated as separate components that are subsequently joined together. For example, bone preparation guide body 30 may be formed integrally with spacer body 12 and/or fulcrum body 14, or may be formed separately from one or both bodies and subsequently fixedly coupled thereto to form a unitary. For example, in different configurations, bone preparation guide body 30 can be welded, bolted, and/or adhesively affixed to spacer body 12 and/or fulcrum body 14 to form a fixed connection between the bone preparation guide body and the spacer body and/or fulcrum body. In either case, spacer body 12, fulcrum body 14, bone preparation guide body 30 may be mechanically interconnected so as to be inseparable during use in a surgical procedure.

In some configurations, bone preparation guide body 30 may be attached to spacer body 12 and/or fulcrum body 14 via a sliding connection. For example, a top end of spacer body 12 may define a rail extending parallel to the widthwise direction of the spacer body. Bone preparation guide body 30 can be mounted on the rail so as to be movable (e.g., slidable) in the medial-to-lateral direction (e.g., parallel to the widthwise direction of spacer body 12). A set screw or other locking feature can be used to lock a specific position of the bone preparation guide body 30 relative to spacer body 12. For example, bone preparation guide body 30 may be operatively connected to an adjuster (e.g., an adjustment knob). The clinician can manipulate the adjuster to control the position of bone preparation guide body 30.

Configuring bone preparation guide body 30 to be movable relative to spacer body 12 and/or fulcrum body 14 can be useful to allow the clinician to move one or more guide surfaces defined by the bone preparation guide body relative to one or more target bones to be cut. This can allow the position of the one or more guide surfaces to be moved depending on the specific anatomy encountered by the clinician during a procedure. The clinician can move the one or more guide surfaces within a bounded range of travel while still having a single, unitary instrument 10 (e.g., optionally graspable by a single handle/hand of the clinician). For example, during the surgical procedure, the clinician may insert combined instrument 10 into joint spaces of the foot, positioning one or more guide surfaces of bone preparation guide body 30 over metatarsal 210 and/or cuneiform 222. The clinician may then adjust the position of bone preparation guide body 30 relative to spacer body 12, e.g., by adjusting the medial-most end of the guide surface to be over the medial-most side of the bone to be cut and/or the lateral-most end of the guide surface to be over the lateral-most side of the bone to be cut. For example, the clinician may position the medial-most end of the guide surface be at or medially-past the medial side of the bone to be cut and/or position lateral-most end of the guide surface be at or lateral-past the lateral side of the bone to be cut. This can position the guide surface so that a bone preparation instrument guided by the guide surface cuts across the entire width of the bone to be cut.

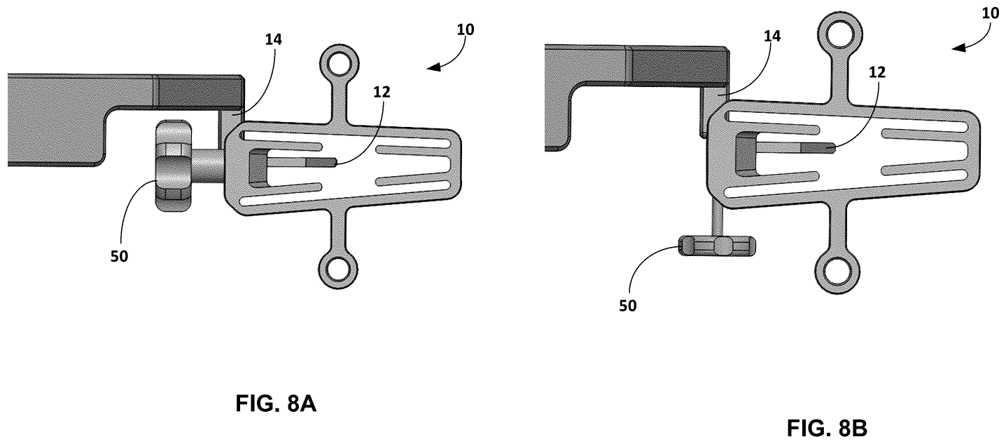

FIGS. 8A and 8B illustrate two example configurations of instrument 10 including an actuator 50. Actuator 50 is illustrated as extending parallel to spacer body 12 in the example of FIG. 8A and perpendicular to spacer body 12 in the example of FIG. 8B. Actuator 50 may be implemented using a screw, knob, ratchet, a gear, and/or other mechanism. Actuator 50 may be configured to move bone preparation guide body 30 relative to spacer body 12 or, in other examples, may merely function to hold a moved position of the bone positioning guide body relative to the spacer body (e.g., with the bone positioning guide body being moved by the hand the clinician and then locked in place using the actuator or locking feature 50).

In some examples, instrument 10 is used as part of a metatarsal realignment procedure in which a metatarsal is realigned relative to an adjacent cuneiform and/or metatarsal in one or more planes, such as two or three planes. Additional details on example bone realignment techniques and devices with which instrument 10 may be used are described in U.S. Pat. No. 9,622,805, titled “BONE POSITIONING AND PREPARING GUIDE SYSTEMS AND METHODS,” filed on Dec. 28, 2015 and issued Apr. 18, 2017, and U.S. Pat. No. 9,936,994, titled “BONE POSITIONING GUIDE,” filed on Jul. 14, 2016 and issued on Apr. 10, 2018, and US Patent Publication No. 2017/0042599 titled “TARSAL-METATARSAL JOINT PROCEDURE UTILIZING FULCRUM,” filed on Aug. 14, 2016. The entire contents of each of these documents are hereby incorporated by reference.

With further reference to FIG. 4, bone preparation guide body 30 is illustrated as extending from a first end positioned over first metatarsal 210 to a second end positioned over medial cuneiform 222. One or both ends of the body can define one or more fixation apertures configured to receive fixation pin(s) for securing bone preparation guide body 30 to one or more bones.

Bone preparation facilitated by bone preparation guide body 30 can be useful, for instance, to facilitate contact between leading edges of adjacent bones, separated by a joint, or different portions of a single bone, separated by a fracture, such as in a bone alignment and/or fusion procedure. A bone may be prepared using one or more bone preparation techniques. In some applications, a bone is prepared by cutting the bone. The bone may be cut transversely to establish a new bone end facing an opposing bone portion. Additionally or alternatively, the bone may be prepared by morselizing an end of the bone. The bone end can be morselized using any suitable tool, such as a rotary bur, osteotome, or drill. The bone end may be morselized by masticating, fenestrating, crushing, pulping, and/or breaking the bone end into smaller bits to facilitate deformable contact with an opposing bone portion.

During a surgical technique utilizing instrument 10, a bone may be moved from an anatomically misaligned position to an anatomically aligned position with respect to another bone. Further, both the end of the moved bone and the facing end of an adjacent end may be prepared for fixation. In some applications, the end of at least one of the moved bone and/or the other bone is prepared after moving the bone into the aligned position. In other applications, the end of at least one of the moved bone and/or the other bone is prepared before moving the bone into the aligned position. In still other applications, the end of one of the moved bone and the other bone is prepared before moving the bone into the aligned position while the end of the opposite facing bone (either the moved bone or the other bone) is prepared after moving the bone into the aligned position.

Movement of one bone relative to another bone can be accomplished using one or more instruments and/or techniques. In some examples, bone movement is accomplished using a bone positioning device, e.g., that applies a force through one or more moving components to one bone, causing the bone to translate and/or rotate in response to the force. This may be accomplished, for example, using a bone positioning guide that includes a bone engagement member, a tip, a mechanism to urge the bone engagement member and the tip towards each other, and an actuator to actuate the mechanism. Additionally or alternatively, bone movement may be accomplished using a compressor-distractor by imparting movement to one bone relative to another bone as the compressor-distractor is positioned on substantially parallel pins, causing the pins to move out of their substantially parallel alignment and resulting in movement of the underlying bones in one plane (e.g., frontal plane, sagittal plane, transverse plane), two or more planes, or all three planes. As yet a further addition or alternative, a clinician may facilitate movement by physically grasping a bone, either through direct contact with the bone or indirectly (e.g., by inserting a K-wire, grasping with a tenaculum, or the like), and moving his hand to move the bone.

When used, the clinician can insert instrument 10 between first metatarsal 210 and second metatarsal 212 and between first metatarsal 210 and medial cuneiform 222 (or other adjacent bones, when not performing a first metatarsal realignment) at any time prior to moving the first metatarsal (e.g., by actuating a bone positioning guide or otherwise manipulating the bone). In one embodiment, the clinician prepares the joint being operated upon to release soft tissues and/or excise the plantar flare from the base of the first metatarsal 210. Either before or after installing an optional bone positioning guide over adjacent bones, the clinician inserts the instrument 10 at the joint between the first metatarsal and the second metatarsal and at the joint between the first metatarsal and medial cuneiform. The clinician can adjust the angular position of fulcrum 12 relative to spacer 14 within the bounded range of rotation set by the instrument before, during, and/or after inserting instrument 10 into the joint spaces. In either case, the clinician can subsequently actuate a bone positioning guide (e.g., when used). As distal portion of first metatarsal can move toward the second metatarsal in the transverse plane to close the IMA, thereby pivoting a proximal portion of the first metatarsal about fulcrum body 14 and reducing the IMA between the first metatarsal and the second metatarsal. The use of fulcrum body 14 can minimize or eliminate base compression between adjacent bones being operated upon.

The clinician can use bone preparation guide body 30 to prepare an end of first metatarsal 210 and an end of medial cuneiform 222. The clinician may prepare the ends of one or both bones before or after moving the first metatarsal in one or more planes (e.g., using bone preparation guide body 30). In either case, the clinician may optionally provisionally fixate the moved position (e.g., by inserting a k-wire or other fixation element) into first metatarsal 210 and an adjacent bone (e.g., second metatarsal 212, medial cuneiform 222). The clinician can remove instrument 10 from the foot, e.g., before or after optionally provisionally fixating. In either case, the clinician may permanently fixate the prepare bone ends, causing the prepared bone ends to fuse together. Example fixation devices that may be used to permanently fixate the joint for fusion include, but are not limited to, plates, staples, screws, pins, and combinations thereof. The one or more fixation devices can be installed across the tarsometatarsal joint from the metatarsal to opposed cuneiform to hold the position of the bones relative to each other for fusion.

In one example technique, after customary surgical preparation and access, a bone preparation instrument can be inserted into the joint (e.g., first tarsal-metatarsal joint) to release soft tissues and/or excise the plantar flare from the base of the first metatarsal 210. Excising the plantar flare may involve cutting plantar flare off the first metatarsal 210 so the face of the first metatarsal is generally planar. This step helps to mobilize the joint to facilitate a deformity correction. In some embodiments, the dorsal-lateral flare of the first metatarsal may also be excised to create space for the deformity correction (e.g., with respect to rotation of the first metatarsal). In certain embodiments, a portion of the metatarsal base facing the medial cuneiform can be removed during this mobilizing step.

An incision can be made and, if a bone positioning instrument is going to be used, one end (e.g., a tip) of a bone positioning guide inserted on the lateral side of a metatarsal other than the first metatarsal 210, such as the second metatarsal 212. The tip can be positioned proximally at a base of the second metatarsal 212 and a third metatarsal 294 interface.

Before or after attaching the optional bone positioning guide, the clinician can insert instrument 10 into the joint. The clinician can position spacer body 12 into the joint space between first metatarsal 210 and medial cuneiform 222 while simultaneously positioning fulcrum body 14 in the joint space between first metatarsal 210 and second metatarsal 212.

In applications utilizing a bone positioning guide, one or more movable features of the bone positioning guide can be moved to reduce the angle (transverse plane angle between the first metatarsal and the second metatarsal) and rotate the first metatarsal about its axis (frontal plane axial rotation). The first metatarsal 210 can be properly positioned with respect to the medial cuneiform 222 by moving a bone engagement member of bone positioning guide with respect to a tip of the bone positioning guide. In some embodiments, such movement simultaneously pivots the first metatarsal with respect to the cuneiform and rotates the first metatarsal about its longitudinal axis into an anatomically correct position to correct a transverse plane deformity and a frontal plane deformity. Other instrumented and/or non-instrumented approaches can be used to adjust a position of first metatarsal 210 relative to medial cuneiform 222. Thus, other applications utilizing instrument 10 may be performed without utilizing bone positioning guide and/or using a bone positioning guide having a different design than the specific example illustrated herein.

In some applications, the end of the first metatarsal 210 facing the medial cuneiform 222 can be prepared with a tissue removing instrument guided by a guide surface of bone preparation guide body 30 (e.g., inserted through a slot defined by a first guide surface and a first facing surface). In some embodiments, the first metatarsal 210 end preparation is done after at least partially aligning the bones, e.g., by actuating bone positioning guide or otherwise moving the first metatarsal but after preparing the end of first metatarsal 210. In other embodiments, the first metatarsal 210 end preparation is done before the alignment of the bones.

In addition to preparing the end of first metatarsal 210, the end of the medial cuneiform 222 facing the first metatarsal 210 can be prepared with the tissue removing instrument guided by a guide surface of bone preparation guide body 30 (e.g., inserted through a slot defined by a second guide surface and a second facing surface). In some embodiments, the medial cuneiform 222 end preparation is done after the alignment of the bones. In yet other embodiments, the medial cuneiform 222 end preparation is done before the alignment of the bones. In embodiments that include cutting bone or cartilage, the cuneiform cut and the metatarsal cut can be parallel, conforming cuts, or the cuts can be angled relative to each other. In some examples, a saw blade can be inserted through a first slot to cut a portion of the medial cuneiform and the saw blade can be inserted through a second slot to cut a portion of the first metatarsal.

Various examples have been described. These and other examples are within the scope of the following claims.

1. A bone cutting and joint realignment instrument, the instrument comprising:
a spacer body configured to be inserted into a joint space between a metatarsal and an opposed cuneiform of a foot; and
a bone preparation guide body affixed to the spacer body with the spacer body extending downwardly from the bone preparation guide body, the bone preparation guide body defining at least one guide surface configured to be positioned over at least one of the metatarsal and the opposed cuneiform;
wherein the bone preparation guide body is affixed to the spacer body via a movable connection.

2. The instrument of claim 1, wherein the bone preparation guide body is configured to move perpendicularly relative to the spacer body via the movable connection.

3. The instrument of claim 1, further comprising an actuator operable to adjust a position of the bone preparation guide body relative to the spacer body.

4. The instrument of claim 1, further comprising a handle connected to the bone preparation guide body.

5. The instrument of claim 4, wherein the bone preparation guide body has a length extending from a first end to a second end, the spacer body is affixed to the bone preparation guide body at the first end, and the handle is connected to the first end of the bone preparation guide body.

6. The instrument of claim 1, wherein the spacer body defines a first portion configured to extend into the joint space between the metatarsal and the opposed cuneiform and a second portion configured to extend above the joint space, the second portion being offset from the at least one guide surface defined by the bone preparation guide body.

7. The instrument of claim 1, wherein the at least one guide surface defined by the bone preparation guide body has a thickness extending from a top end to a bottom end, the spacer body has a length extending from a top end to a bottom end, and the top end of the spacer body is approximately coplanar with or located vertically below the bottom end of the at least one guide surface.

8. The instrument of claim 1, wherein the bone preparation guide body comprises at least a first guide surface configured to be positioned over the metatarsal and a second guide surface configured to be positioned over the opposed cuneiform, the first and second guide surfaces being separated from each other by a distance configured to span a tarsometatarsal joint.

9. The instrument of claim 8, wherein the first guide surface and the second guide surface each have a thickness less than 2 mm.

10. The instrument of claim 8, wherein the bone preparation guide body further comprises:
a first facing guide surface spaced apart from the first guide surface to define a first cutting slot therebetween, and
a second facing surface spaced apparent from the second guide surface to define a second cutting slot therebetween.

11. The instrument of claim 10, wherein the first facing guide surface and the second facing guide surface each have a thickness less than 2 mm.

12. The instrument of claim 1, further comprising a fulcrum body rotatably coupled to the spacer body within a bounded range of rotation of less than 90 degrees, the fulcrum body being configured to be inserted in an intermetatarsal space between the metatarsal and an adjacent metatarsal.

13. The instrument of claim 12, wherein the spacer body and the fulcrum body define an angular intersection therebetween, and the fulcrum body is rotatably coupled to the spacer body to allow the angular intersection to adjust from a minimum of approximately 90 degrees to a greater angle.

14. The instrument of claim 13, wherein the bounded range of rotation is approximately 25 degrees or less, and the angular intersection is within a range from 90 degrees to 115 degrees.

15. A method comprising;
inserting a bone cutting and joint realignment instrument into a joint space of the foot, the bone cutting and joint realignment instrument comprising a spacer body and a bone preparation guide body movably affixed to the spacer body, wherein inserting the bone cutting and joint realignment instrument into the joint space of the foot comprises inserting the spacer body between a metatarsal and an opposed cuneiform of the foot and concurrently positioning at least one guide surface defined by the bone preparation guide body over one or both of the metatarsal and opposed cuneiform;
adjusting a position of the at least one guide surface relative to the spacer body via a movable connection;
preparing an end of the metatarsal by guiding a bone preparation instrument along the at least one guide surface defined by the bone preparation guide body;
preparing an end of the opposed cuneiform by guiding the bone preparation instrument along the at least one guide surface defined by the bone preparation guide body;
moving the metatarsal relative to an adjacent metatarsal; and
applying at least one fixation device across a tarsometatarsal joint between a prepared end of the metatarsal and a prepared end of the opposed cuneiform.

16. The method of claim 15, wherein adjusting the position of the at least one guide surface relative to the spacer body via the movable connection comprises moving the at least one guide surface perpendicularly relative to the spacer body via the movable connection.

17. The method of claim 15, wherein adjusting the position of the at least one guide surface relative to the spacer body comprises operating an actuator to adjust a position of the bone preparation guide body relative to the spacer body.

18. The method of claim 15, wherein adjusting the position of the at least one guide surface relative to the spacer body comprises moving the bone preparation guide body relative to the spacer body in a medial-to-lateral direction.

19. The method of claim 15, wherein:
the at least one guide surface defined by the bone preparation guide body comprises a first guide surface configured to be positioned over the metatarsal and a second guide surface configured to be positioned over the opposed cuneiform, and
positioning the at least one guide surface defined by the bone preparation guide body over one or both of the metatarsal and opposed cuneiform comprises positioning the first guide surface over the metatarsal and the second guide surface over the opposed cuneiform.

20. The method of claim 15, wherein:
the metatarsal is a first metatarsal;
the opposed cuneiform is a medial cuneiform; and
preparing the end of the metatarsal and preparing the end of the opposing cuneiform comprises preparing one or both of the end of the metatarsal and the end of the opposed cuneiform after moving the metatarsal relative to the adjacent metatarsal.
